(19) United States
(12) Patent Application Publication
(10) Pub. No.: US 2004/0053318 A1
(43) Pub. Date: Mar. 18, 2004

(54) Preservation of RNA and reverse transcriptase during automated liquid handling

(76) Inventors: Diana R. McWilliams, Bonne Terre, MO (US); Charles W. Bolten, Kirkwood, MO (US)

Correspondence Address: Gardere Wynne Sewell LLP, Suite 3400, 1000 Louisiana, Houston, TX, 77002, US

(21) Appl. No.: 10/661,284

(22) Filed: Sep. 12, 2003

Related U.S. Application Data
(60) Provisional application No. 60/411,174, filed on Sep. 17, 2002.

(51) Int. Cl. C12Q 1/68; C12M 1/34

(52) U.S. Cl. 435/6; 435/287.2

(57) ABSTRACT

The present invention is a metal block for use in a high throughput RNA laboratory comprising a plurality of wells, each well having an open cylindrical upper end and a closed conical lower end. Each well is design to accommodate a biological sample receptacle. The receptacle has substantially the same shape as the well, thereby maintaining the temperature of a biological sample in the receptacle during sample set up and prior to polymerase chain reaction. Use of the metal block in an automated liquid handling device provides an improvement to liquid handling systems currently available.

### CROSS-REFERENCE TO RELATED APPLICATIONS

The present application claims priority under Title 35, United States Code, § 119(e)(1) of U.S. Prov. Pat. App. Ser. No. 60/411,174, filed Sep. 17, 2002.

### BACKGROUND

A key area of pharmaceutical research is the determination of genetic expression. In vivo experimentation of pharmacological products mandates an accurate analysis of the cellular function and gene expression to determine efficacy and safety. The expression of a particular gene is often an indicator of the efficacy of the drug product.

The polymerase chain reaction (“PCR”) has revolutionized genetic research by providing a rapid means of amplifying and subsequently identifying specific nucleic acid sequences from complex genetic samples without the need for time-consuming cloning, screening and nucleic acid purification protocols. PCR was originally disclosed and claimed by Mullis et al. in U.S. Pat. Nos. 4,683,195, 4,683,202, and 4,965,188, hereby incorporated by reference. Since that time, considerable advances have been made in the reagents, equipment and techniques available for PCR. These advances have increased both the efficiency and utility of the PCR reaction, leading to its adoption in an increasing number of different scientific applications and situations.

The earliest PCR techniques were directed toward qualitative and preparative methods rather than quantitative methods. PCR was used to determine if a given DNA sequence was present in any quantity at all or to obtain sufficient quantities of a specific nucleic acid sequence for further manipulation. Originally, PCR was not typically employed to measure the amount of a specific DNA or RNA present in a sample. Only in recent years has quantitative PCR come to the forefront of nucleic acid research.

While DNA is necessary for PCR analysis, in testing the efficacy and safety of drugs, it is the mRNA that is the most accurate indicator of gene expression. There are many steps in the pathway leading from DNA to protein and all of them can in principle be regulated. A cell controls the proteins its makes by: 1) controlling when and how often a given gene is transcribed (transcriptional control), 2) controlling how the primary RNA transcript is spliced or otherwise processed (RNA processing control), 3) selecting which completed mRNAs in the cell nucleus are exported to the cytoplasm (RNA transport control), 4) selecting which mRNAs in the cytoplasm are translated by ribosomes (translational control), 5) selectively destabilizing certain mRNA molecules in the cytoplasm (MRNA degradation control), or 6) selectively activating, inactivating or compartmentalizing specific protein molecules after they have been made (protein activity control). Molecular Biology of the Cell, 3^{rd} Ed. at 403. Although all of these steps involved in expressing a gene can in principle be regulated, for most genes, transcriptional controls are paramount and the initiation of RNA transcription is the most important point of control. Id. Therefore, mRNA is purified and cDNA clones produced to measure gene expression in the experimentation of pharmacological products.

Amplification of RNA into cDNA clones is accomplished by including a reverse transcription step prior to the start of PCR amplification. Reverse transcriptase (“RT”) is a DNA polymerase used to synthesize a cDNA strand using an MRNA template and primer, and is often used in conjunction with PCR in order to measure gene expression. This process is known as RT-PCR. By purifying mRNA, producing cDNA and amplifying the cDNA, gene expression is measured.

In a one-step RT-PCR process, reverse transcriptase, Taq polymerase, primers, dNTPs and MRNA are added to the same tube and reverse transcription and amplification occur without further removal or addition of reagents. In two-step RT-PCR, reverse transcriptase, MRNA, dNTPs, and primers are used to make cDNA. The cDNA may be transferred to a new tube and primers, dNTPs, probes and Taq polymerase are then added together to amplify the DNA. The two-step protocol is prone to contamination because of the need to expose the samples to air while adding reagents.

Moreover, the reverse transcriptase is a temperature sensitive enzyme that begins to degrade above approximately 10° C. While optimal activity of the enzyme occurs at 37 to 48° C., the enzyme quickly degrades at this temperature. Even though reverse transcription is performed between 37 to 48° C., the reverse transcriptase looses activity during prolonged periods of elevated temperature. Reverse transcriptase maintains activity for at least 8 hours when stored at 4° C. However, activity may be lost within 30 minutes at a temperature of 48°C.

Once at room temperature, mRNA may denature if not used immediately as RNA degrades when exposed to heat or high pH. RNA degradation by alkaline hydrolysis is accelerated by heat. While RNase inhibitors may be added to protect the MRNA, RNase contamination may occur and degrade the mRNA. If RNA is degraded, an inaccurate analysis may result. Hence, maintaining RNA at a low temperature minimizes degradation.

Also, at room temperature, taq polymerase activity may begin prior to the start of PCR. When this occurs, the yield and specificity of PCR is decreased at least partially due to the priming (or mis-priming) of sequences. Hence, premature taq polymerase activity provides inaccurate results in the analysis of genetic expression.

In order to analyze the sample, the RNA must be purified, reagents transferred into the biological sample receptacle, and the nucleic acid sequence amplified It is important that the contamination and degradation of mRNA be minimized. Hence, following purification of the mRNA or DNA, an automated liquid handling device is often used to add reagents to the biological sample receptacle reactions to maintain accuracy and eliminate repetitive injury to researchers.

Automated liquid handling devices used in laboratories increase the sample throughput and decrease pipetting error as compared with a human being. Examples of such devices include the Beckman Biomek®, the Qiagen 8000, 3000 or 9600, the Gilson Constellation® 1200 Liquid Handler, the Zymark Sciclone ALH, Staccato® Plate Replication Workstation, RapidPlate® 96/384 Microplate Pipetting Workstation, and the Robbins Scientific Tango Liquid Handling System. These devices are able to transfer reagents from one location to another according to a pre-programmed pattern.

Typically, the automated liquid handling device has a refrigerated table that maintains the temperature of the sample. However, the refrigerated table is not satisfactory for maintaining the sample at a sufficient temperature to preserve the activity of the enzyme and avoid degradation of mRNA. Hence, reverse transcriptase inactivation, mRNA denaturation, and Taq activation may begin before amplification cycles (94° C. for 2 to 10 minutes) tainting the expression results.

Moreover, racks for holding biological sample receptacles, such as microtubes and 96- and 384-well plates upon an automated liquid handling device, are routinely plastic with a cylindrical well shape. The racks are not designed to maintain low temperature. Therefore, the cooling effect of the refrigerated table is dissipated and certain enzymes added to the sample receptacles lose activity. In addition, most available sample racks are not designed for use on an automated liquid handling device. For example, aluminum racks such as the benchtop working racks for the Stratagene StrataCooler® are first chilled at 4° C. for one hour and then placed in a plastic outer cooler that has been frozen at −20 to −25° C. for 24 hours. This type of device is simply a cooler and is not subject for use in an automated liquid handling device.

Other devices designed to provide temperature control of a sample, such as the plates of thermal cyclers, usually contain fluid flow channels through which to pass a fluid of a given temperature or a thermoelectric heat pump to control the temperature of the sample (U.S. Pat. Nos. 5,333,675 and 5,038,852). Even with the cumbersome equipment set up, heat transfer is likely to occur through the well containing the sample receptacle. Still other devices such as centrifuge rotors require a refrigeration system that maintains the entire chamber at a given temperature (U.S. Pat. No. 4,833,891).

A need exists, therefore, for a low cost, low maintenance, simple-to-use device that maintains the entire contents of the biological sample receptacle at a given temperature upon an automated liquid handling device.

### SUMMARY OF THE INVENTION

The present invention is a metal block for use in a high throughput RNA laboratory comprising a plurality of wells. Each well has an open cylindrical upper end and a closed conical lower end. Each well is design to accommodate a biological sample receptacle. The receptacle has substantially the same shape as the well, thereby maintaining the temperature of a biological sample in the receptacle during sample set up and prior to polymerase chain reaction. Use of the metal block in an automated liquid handling device provides an improvement to liquid handling systems currently available.

The metal block is particularly useful for high throughput RNA analysis of a biological sample where the biological sample is inserted into the biological sample receptacles positioned in the wells of the metal block by the automated liquid handling device. In a nucleic acid amplification device, the sample is then caused to undergo reverse transcriptase polymerase chain reaction to determine the presence of RNA or DNA.

The subject invention also provides a method of preparing and handling a biological sample for high throughput RNA analysis including the steps of liquefying or pulverizing a biological sample and inserting the sample into a receptacle placed in the metal block. The metal block is first chilled and then fixed into position on an automated liquid handling device. The metal block and the liquefied biological sample temperature is maintained on the liquid handling device and reagents are added to the liquid biological sample for reverse transcriptase and polymerase chain reaction analysis.

The subject invention also is an improved automated liquid handling device for genetic analysis of biological samples. The typical handling device is adapted to transfer, dispense and aspirate liquid from one location to another automatically and is capable of a wide range of bioanalytical procedures including sample pipetting, serial dilution, reagent additions, mixing reaction timing and similar known manual procedures. The typical handling device includes table for supporting microtiter plates and other biological sample receptacles, a pod for transferring fluid to a well located on the table and a means for moving the pod relative to the table between selected locations on said table. The improvement to the liquid handling device is use of the metal block having a plurality of wells, each well having an open cylindrical upper end and a closed conical lower end. Each well accommodates a biological sample receptacle having substantially the same shape as the well and the temperature of a biological sample in the receptacle during sample set-up and prior to polymerase chain reaction analysis is maintained.

### BRIEF DESCRIPTION OF SEVERAL VIEWS OF THE DRAWINGS

For better understanding of the invention and to show by way of example how the invention may be carried into effect, reference is now made to the detail description of the invention along with the accompanying figures in which corresponding numerals in the different figures refer to corresponding parts and in which:

FIG. 1A is a perspective view of the metal block suitable for polypropylene tubes.

FIG. 1B is a perspective view of the metal block suitable for a 96 well format.

FIG. 2 is an exploded view of the metal block and biological sample receptacles.

FIG. 3 is a cross-sectional view of the metal block.

FIG. 4 is a perspective view of a liquid handling device suitable for use in connection with the subject invention.

FIGS. 5 through 11 represent data obtained and analyzed in Example 1.

FIGS. 12 through 19 represent data obtained and analyzed in Example 2.

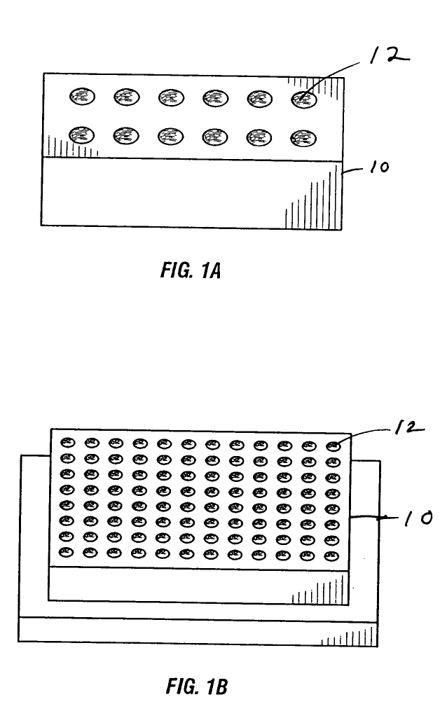

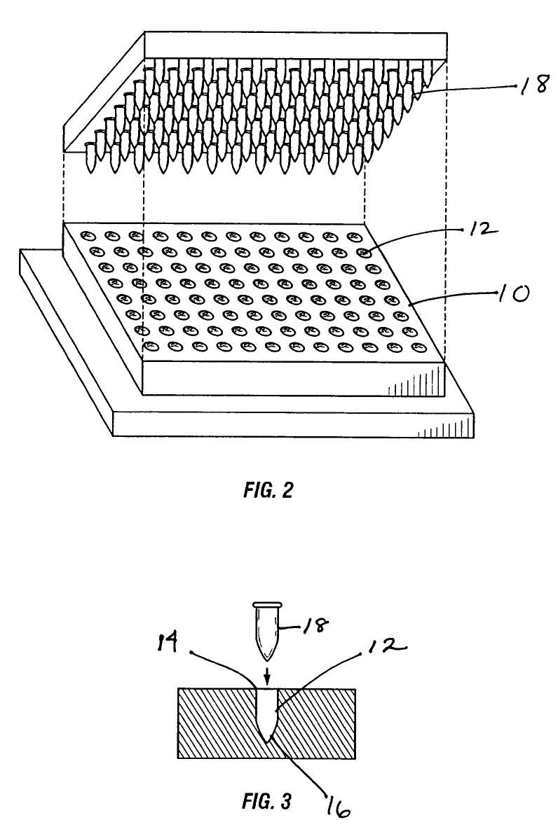

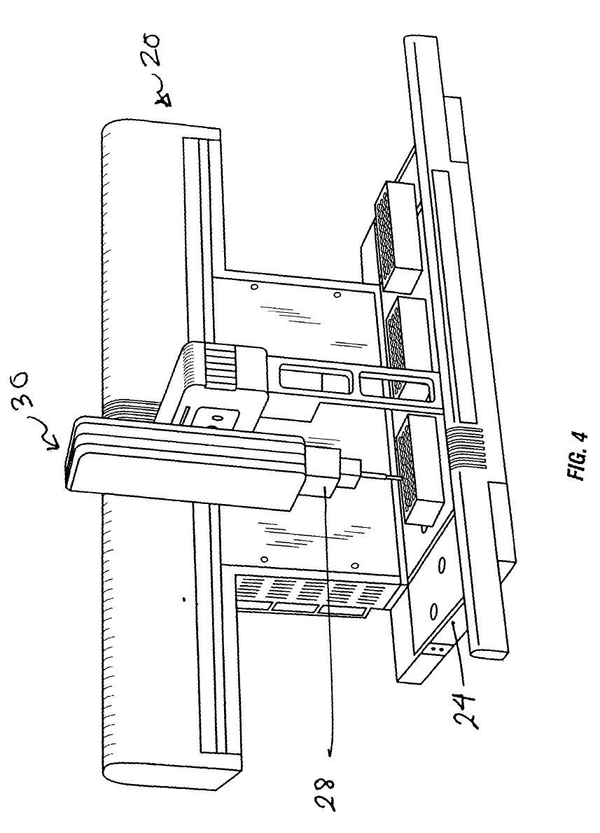

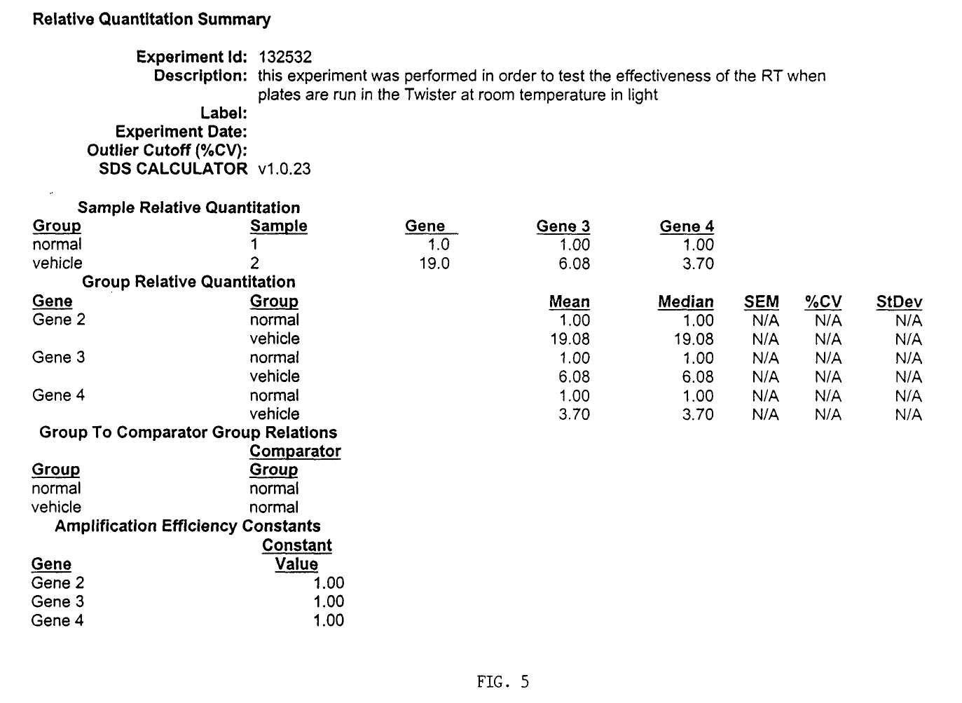

### DETAILED DESCRIPTION

As shown in the figures, the present invention is a metal block 10 for use in a high throughput RNA laboratory comprising a plurality of wells 12. Each well 12 has an open cylindrical upper end 14 and a closed conical lower end 16. Each well 12 is designed to accommodate a biological sample receptacle 18. The receptacle 18 has substantially the same shape as the well, thereby maintaining the temperature of a biological sample in the receptacle during sample set up and prior to polymerase chain reaction. Use of the metal block with an automated liquid handling device 20 and for genetic analysis of biological samples provides an improvement to liquid handling systems currently available.

The metal block 10 is particularly useful for high throughput RNA analysis of a biological sample in combination with an automated liquid handling device. Here, the biological sample is inserted into the biological sample receptacle 18 as held by the wells 12 of the metal block 10 in the automated liquid handling device 20. Subsequently, reverse transcriptase polymerase chain reaction is used to determine the presence of RNA or DNA in the sample via a nucleic acid amplification machine.

The subject invention also is an improved automated liquid handling device 20 for genetic analysis of biological samples. The handling device 20 controls dispensing, aspirating and transferring of liquid from a first microtiter plate well or other biological sample receptacle to a second microtiter plate well or other second biological sample receptacle. The automated liquid handling device is capable of functioning with test tubes, freezing vials, reservoirs and other wet chemistry containers. The improvement to the liquid handling device comprises use of the metal block 10 comprising a plurality of wells 12 where each well 12 has an open cylindrical upper end 14 and a closed conical lower end 16. Each well 12 accommodates a biological sample receptacle 18 having substantially the same shape as the well 12. The biological sample and reagents are pipeted into the receptacle 18 and the temperature of a biological sample during sample set-up and prior to polymerase chain reaction analysis is maintained.

Also, a method of handling a liquid biological sample in a high throughput RNA laboratory is provided. Such method includes the steps of chilling the metal block, inserting the biological sample receptacle into the metal block, positioning the metal block onto an automated liquid handling device and transferring the biological sample into biological sample receptacle in the metal block for polymerase chain reaction analysis.

The metal block of the subject invention is preferably made of aluminum, but may be made of other materials including, but not limited to, copper, gold, or silver. Any material with having high thermal conductivity may be suitable for use in the present invention. The metal block is designed to maintain sample temperature of 0 to 10° C.

The suitable biological sample receptacle includes polypropylene tubes, thermal cycler tubes, a 96 well plate, or a 384-well plate. Biological sample receptacles may be made of plastic or glass. Frequently, biological sample receptacles are plastic and are made of polypropylene or polycarbonate. Thin-walled tubes and plates are preferred as they allow rapid and consistent heat transfer. Tube volume capacity may range from approximately 0.2 milliliters to 1.7 milliliters. Volume capacities of individual microplate tubes vary from approximately 0.2 milliliters in a 96 well format to approximately 0.04 milliliters for the 384 well format.

A biological sample as used herein may be any composition comprising RNA, DNA or genetic sequences created using RNA or DNA from any one or more of the tissues that make up an animal or tissue culture. The tissue from which the RNA originated may include, but are not limited to, epithelial, connective, muscular, and nerve tissues.

To purify a nucleic acid sequence or mRNA, a sample is first collected and liquefied or pulverized. It is important that RNA purification is done by a method that minimizes degradation. The researcher analyzing the results of gene expression must collect and analyze animal tissues as quickly as possible, beginning at the time the animal is euthanized and the organs harvested.

MRNA is subsequently purified using one of a number of methods or devices including a automated nucleic acid workstation such an ABI Prism® 6700. Other devices for purification include but are not limited to the Qiagen BioRobot 9604 or 8000. The technician may also purify the RNA or DNA without using a nucleic acid workstation using alternative purification methods including, but not limited to, glass fiber filter systems such as RNeasy by Qiagen, RNaqueous technology from Ambion, or Absolutely RNA Microprep Kit from Stratagene. RNA may also be purified through precipitation reactions using phenol based products, isopropyl alcohol and lithium chloride. Also, available is a product known as Nucleopin by BD Biosciences.

Following purification of the RNA or DNA, reagents are added to the biological sample in the biological sample receptacle 18 so that the RT-PCR or PCR reaction may occur. Commonly used reverse transcriptases include, but are not limited to, avian myeloblastosis virus (AMV), or Moloney murine leukemia virus (MMLV or MuLV). MMLV and MuLV have lower RNase H activities than AMV but AMV is more stable at higher temperatures. As an alternative, some thermostable DNA polymerases such as Thermus thermophilus DNA polymerase have reverse transcriptase activity in the presence of manganese, allowing for the use of only one enzyme for reverse transcription and polymerase chain reaction. If bicine buffer with manganese is used, intermediate additions between reverse transcription and amplification are not needed and stability at elevated temperatures is not a concern. However the presence of manganese may reduce the fidelity of nucleotide incorporation. Therefore, this method is not suitable for a high throughput RNA analysis. As described in more detail below, other reagents may include, but are not limited to, oligonucleotide primers, a thermostable DNA polymerase and an appropriate reaction buffer such as 500 mM KCl, 100 mM Tris-HCl, 0.1 mM EDTA.

Automated liquid handling devices are often used in laboratories to increase the sample throughput and decrease pipetting error as compared with a human being. These devices are able to transfer reagents from one location to another according to a pre-programmed pattern. The refrigerated table designed to maintain sample temperature table is not satisfactory for maintaining the sample at a sufficient temperature to preserve the activity of the enzyme.

The Beckman Biomek® 2000 is an example of one such device. The Biomek 2000 is an automated liquid handling workstation capable of programmed tasks such as sample pipetting, serial dilution, reagent additions, mixing, reaction timing and similar known manual procedures. The Biomek® 2000 is adapted to aspirate liquid from one location to dispense the liquid in another location automatically in accordance with user programmed instructions. In this liquid handling system, microtiter plates, tip support plates, and troughs are supported in a table attached to the laboratory workstation base. Movement of the table is provided by a motor means causing the table to reciprocally move in at least one axis. A modular pod suspended above the table has an arm attached at one end for movement up and down a vertically extending tower rising from the base of the workstation. The pod is capable of motion along the arm in at least a second axis that is perpendicular to the first axis of movement of the support table. The arm moves up and down in a third direction perpendicular to both the first and second directions.

As more fully described in U.S. Pat. Nos. 5,104,621 and 5,108,703, incorporated herein by reference, the pod is connected with and supports a fluid dispensing, aspirating and transferring means. In the Biomek® 2000, a fluid dispensing pump is connected to the pod by fluid conduits to provide pipetting, dispensing, and aspirating capability. Fluid is dispensed using interchangeable modules of one or more nozzles. The nozzles have pipettor tips affixed to them that are automatically picked up and ejected by the pod.

As shown in FIG. 4, this automated liquid handling device has a table 24, a pod 28 for transferring fluid to a well located on the table 24 and a means 30 for moving the pod relative to the table between selected locations on table 24. The table 24 acts as a surface for supporting the metal block, biological sample receptacles, reagent reservoirs and pipettor tips. The pod 28 is capable of movement horizontally and vertically. The temperature of the table 24 is controllable and is achieved through the use of one or more circulating water baths.

As with many liquid handling devices, the Biomek® 2000 liquid handling device is capable of being programmed to maintain the table at a given temperature and to pipet all reagents required for a given assay into a biological sample receptacle. The device software allows the user to specify the location of the aspiration, dispensation and mixing, what type of labware the liquid is being aspirated from and into and the volume and height of the aspiration and dispensation.

Other devices that may be used include, but are not limited to, the Qiagen 8000, 3000 or 9600, the Gilson Constellations 1200 Liquid Handler, the Zymark Sciclone ALH, Staccato® Plate Replication Workstation, or RapidPlate® 96/384 Microplate Pipetting Workstation. The Qiagen BioRobot 8000 is a nucleic acid purification and liquid handling workstation. It has robotic handling, automated vacuum and a buffer delivery system. Sample receptacles and reagent troughs are present on a platform and an 8 channel pipetting system performs high-speed dispensing. The Qiagen BioRobot 3000 is an automated liquid handling and sample processing workstation. It allows the integration of other hardware, such as cyclers or spectrophotometers. It has fully automated plate processing by transferring labware to various positions on and off of the worktable, as well as temperature control, small volume liquid handling and customizable processing parameters. The Qiagen BioRobot 9600 is an automated workstation for nucleic acid purification, reaction set-up, PCR product clean-up, agarose-gel loading and sample rearray and has a worktable and programmable pipetting mechanism. The Gilson Constellation 1200 Liquid Handler has a bed that can hold up to 12 microplates, a robotic gripper arm, capability to dispense nanoliter volumes and an optional heating and cooling recirculator. The Zymark Sciclone ALH Workstation has a 20 position deck, bulk dispensing capabilities to microplates by syringe or peristaltic pump and can pipet using a single channel, 8 channel, 12 channel or 96 channel head. The Robbins Scientific Tango Liquid Handling System comprises a worktable and automated aspiration and dispensing of liquid in a 96 or 384 well format. These devices are able to transfer reagents from one location to another according to a pre-programmed pattern and may be suitable for use in connection with the present invention.

In the subject invention, a biological sample for high throughput RNA analysis is prepared by liquefying or pulverizing the biological sample; then extracting RNA by a variety of methodology. The metal block 10 having been previously refrigerated or frozen is fixed into position on an automated liquid handling device 20. Biological sample receptacles 18 are then inserted into the metal block 10. As the temperature of the liquefied biological sample is maintained, reagents are added to the liquid biological sample for polymerase chain reaction analysis. Reagents are added into the biological sample receptacles 18 by the automated liquid handling device. The biological sample receptacles are then either moved by robot or manually to a sequence detection system where the reverse transcription, polymerase chain (RT-PCR) reaction amplification and analysis occur.

PCR amplification of a specific DNA segment, referred to as the template, requires that the nucleotide sequence of at least a portion of each end of the template be known. From the template, a pair of corresponding synthetic oligonucleotide primers (“primers”) can be designed. The primers are designed to anneal to the separate complementary strands of template, one on each side of the region to be amplified, oriented with its 3′ end toward the region between the primers. The PCR reaction needs a DNA template along with a large excess of the two oligonucleotide primers, a thermostable DNA polymerase, dNTPs and an appropriate reaction buffer.

To effect amplification, the mixture is denatured by heat to cause the complementary strands of the DNA template to disassociate. The mixture is then cooled to a lower temperature to allow the oligonucleotide primers to anneal to the appropriate sequences on the separated strands of the template. Following annealing, the temperature of the reaction is adjusted to an efficient temperature for 5′ to 3′ DNA polymerase extension of each primer into the sequences present between the two primers. This results in the formation of a new pair of complementary strands. The steps of denaturation, primer annealing and polymerase extension can be repeated many times to obtain a high concentration of the amplified target sequence. Each series of denaturation, annealing and extension constitutes one “cycle.” There may be numerous “cycles.” The length of the amplified segment is determined by the relative positions of the primers with respect to each other, and therefore, this length is a controllable parameter. By virtue of the repeating aspect of the process, the method is referred to as the “polymerase chain reaction” (hereinafter “PCR”).

As the desired amplified target sequence becomes the predominant sequence in terms of concentration in the mixture, this sequence is said to be PCR amplified. With PCR, it is possible to amplify a single copy of a specific target DNA sequence to a level detectable by several different methodologies. These methodologies include ethidium bromide staining, hybridization with a labeled probe, incorporation of biotinylated primers followed by avidin-enzyme conjugate detection, and incorporation of 32P-labeled deoxynucleotide triphosphates such as Dctp or Datp into the amplified segment.

The development of real-time PCR, also known as kinetic PCR, has provided an improved method for the quantification of specific nucleic acids. In real-time PCR, cycle-by-cycle measurement of accumulated PCR product is made possible by combining thermal cycling and fluorescence detection of the amplified product in a single instrument. Because the product is measured at each cycle, product accumulation can be plotted as a function of cycle number. The exponential phase of product amplification is readily determined and used to calculate the amount of template present in the original sample. A number of alternative methods are currently available for real-time PCR.

The original protocol developed by Grossman et al. (U.S. Pat. No. 5,470,705, hereby incorporated by reference) used radioactive labels on the probes but further refinements of the method have focused on self-quenching fluorescent probes. Originally, separation of the amplified products by electrophoresis or other methods was used to measure and calculate the amount of released label. This added time-consuming steps to the analysis. Furthermore, this end-stage analysis of the reactions cannot be readily applied to real-time PCR.

In one current method, fluorogenic exonuclease probes for the real-time detection of PCR products are used. This type of technology is captured in the ABI Prism® 7700 Sequence Detection System and disclosed in Livak et al (U.S. Pat. No. 5,538,848 hereby incorporated by reference). In a modification of an existing method utilizing radioactive labels, fluorogenic exonuclease probes are designed to anneal to sequences between the two amplification primers but contain one or more nucleotides that do not match at the 5′ end. The nonmatching nucleotides are linked to a fluorescence donor. A fluorescence quencher is positioned typically at the end of the probe. When the donor and quencher are in the same vicinity, the quencher prevents the fluorescence donor from emitting light.

Traditional fluorescence quenchers absorb light energy emitted by an excited reporter molecule and release this energy by fluorescing at a higher wavelength. Increased sensitivity in real-time detection can be achieved with dark quenchers such as dabcyl or the developed Eclipse Quencher from Epoch Biosciences, Inc. The dark quenchers absorb fluorescent energy but do not fluoresce themselves, thus reducing background fluorescence in the sample. The dark quencher works effectively against a number of red-shifted fluoropores such as FAM, Cy3 and Tamra due to its broader range of absorbance over dabcyl (400-650 nm versus 360-500 nm respectively) and is thus better suited to multiplex assays.

The sensitivity of real-time PCR can also be augmented through the use of minor groove binders (“MGBs”) (also from Epoch Biosciences, Inc.), which are certain naturally occurring antibiotics and synthetic compounds able to fit into the minor groove of double-stranded DNA to stabilize DNA duplexes. The minor groove binders can be attached to the 5′ end, 3′ end or an internal nucleotide of oligonucleotides to increase the oligonucleotide's temperature of melting, i.e., the temperature at which the oligonucleotide disassociates from its target sequence and hence creates stability. The use of MGBs allows for the use of shorter oligonucleotide probes as well as the placement of probes in AT-rich sequences without any loss in oligonucleotidal specificity, as well as better mismatch discrimination among closely related sequences. Minor groove binders may be used in connection with dark quenchers or alone.

Thermus aquaticus (taq) DNA polymerse used for the PCR amplification has the ability to cleave unpaired nucleotides off of the 5′ end of DNA fragments. In the PCR reaction, the fluorogenic probe anneals to the template (the nucleotide sequence of interest in a sample). An extension of both primers and the probe occurs until one of the amplification primers is extended to the probe. Taq polymerase then cleaves the nonpaired nucleotides from the 5′ end of the probe, thereby releasing the fluorescence donor. Once it is physically separated from the quencher, the fluorescent donor can fluorescence in response to light stimulation. Because of the role of taq polymerase in this process, these probes are often referred to as TaqMan® probes. As more PCR product is formed, more fluorescent donors are released, allowing the formation of the PCR product to be measured and plotted as a function of cycle time. The linear, exponential phase of the plot can be selected and used to calculate the amount of nucleotide in the sample. The development of these self-quenching fluorescent probes was a considerable advancement in quantitative PCR. Numerous improved self-quenching probes and methods for the use thereof have been subsequently reported in U.S. Pat. Nos. 5,912,148, 6,054,266 (Kronick et al.) and U.S. Pat. No. 6.130,073 (Eggerding).

The LightCycler® uses hybridization instead of exonuclease cleavage to quantify the amplification reaction. This method also adds additional fluorogenic probes to the PCR amplification. However, unlike the TaqMan® system, fluorescence increases in this system when two different fluorogenic probes are brought together on the same template by extension or hybridization, allowing resonance energy transfer to occur between the two probes.

Other systems are also available. The Amplifluor® primers produced by Intergen® are hairpin oligonucleotides, which form hairpins when they are single-stranded, which bring a fluorescence donor and quencher into close proximity. When the primers are incorporated into a double stranded molecule, the hairpins are straightened, which separates the donor and quencher to cause an increase in fluorescence. Other applications use intercalating dyes, which only associate with double stranded DNA. As more double stranded DNA is generated by the reaction, more fluorescence is observed as more dye becomes associated with DNA. Regardless of the method used, the end result is the same, a plot of fluorescence versus cycle number. Further analysis of this data is then used to derive quantitative values for the RNA present in the samples. Hence, amplified segments created by the PCR process are efficient templates for subsequent PCR amplifications leading to a cascade of further amplification.

The amplification of nucleic acid sequences may occur within and be analyzed by a sequence detection system, such as the ABI Prism® 7900. The sequence detection system is able to vary reaction conditions to optimize amplification of a nucleic acid sequence. The system can analyze the amount of a given nucleic acid sequence present using any number of fluorescent probes, a fluorescence detection mechanism and system software. Other devices that may be used to provide temperature cycling with or without detection capabilities including but are not limited to a Roche Applied Science LightCycler®, BioRad iCycler, MJ Research Opticon, Corbett Rotorgene, and Stratagene Mx4000 Multiplex Quantitative PCR System. A fluorimeter and analysis program may be used in conjunction with devices in which these functions are not integrated. The sequence detection system is able to vary reaction conditions to optimize amplification of a nucleic acid sequence. The system can analyze the amount of a given nucleic acid sequence present using any number of fluorescent probes, a fluorescence detection mechanism and sequence detection system software.

### EXAMPLE 1

### Room Temperature Stability Study

This experiment was prepared to determine the stability of a TaqMan® plate at room temperature if the AB One-Step RT-PCR Master Mix Kit is used. Here, both the reverse transcriptase and the Taq polymerase are added simultaneously to generate a cDNA first and then the cDNA is amplied without reopening the tube.

A total of eight plates were pippeted by the Biomek 2000 robot. Each plate was identical using the same primer-probe sets and the same total RNA template. AB One-Step RT-PCR Master Mix was the reagent used. This allows cDNA and amplification of said cDNA to occur in one tube or well position without reopening the tube or well position. Plates were loaded onto the Zymark Twister and sat at room temperature until each plate was autoloaded into the ABI 7900 for a standard real-time run which spanned two hours and was equivalent to our routine lab format used for a single plate. Table 1 lists the contents of the various plates and the amount of time each plate was maintained at room temperature before the test run.

TABLE 1

| Hours @ room temp before | Plate 2 | Plate 3 | Plate 4 | Plate 5 | Plate 6 | Plate 7 | Plate 8 |
|---|---|---|---|---|---|---|---|
| run | 2 hours | 4 hours | 6 hours | 8 hours | 10 hours | 12 hours | 14 hours |
| Ct Gene 1 Unstimulated Cells | 17.8 | 17.7 | 17.6 | 17.6 | 17.5 | 17.6 | 17.6 |
| Ct Gene 1 Stimulated Cells | 17.8 | 17.6 | 17.6 | 17.5 | 17.5 | 17.4 | 17.5 |
| Ct Gene 2 Unstimulated Cells | 32.6 | 32.7 | 32.7 | 32.5 | 34.3 | 34.5 | 34.9 |
| Ct Gene 2 Stimulated Cells | 30.7 | 30.8 | 30.6 | 30.8 | 32 | 32.1 | 32.2 |
| Ct Gene 3 Unstimulated Cells | 22.2 | 22.2 | 22.2 | 22.2 | 23.2 | 23.3 | 23.3 |
| Ct Gene 3 Stimulated Cells | 17.8 | 17.8 | 17.9 | 17.8 | 18.9 | 18.9 | 19 |
| Ct Gene 4 Unstimulated Cells | 21 | 21 | 21 | 21 | 22.1 | 22.1 | 22.2 |
| Ct Gene 4 Stimulated Cells | 18.4 | 18.3 | 18.3 | 18.3 | 19.4 | 19.4 | 19.5 |

Data from each plate was analyzed with baseline set from Cycle 3 to Cycle 14 and Cycle Threshold set at 0.1. Plate 1 was eliminated due to technical error. Data from Plate 2 to Plate 8 is presented as Ct data. Table 2 is a spreadsheet of such data.

TABLE 2
Plate 2

| | | | |
|---|---|---|---|
| 40 | 40 | 40 | 40 |
| 40 | 40 | 40 | 40 |
| 17.807295 | 17.802525 | 22.195833 | 22.12981 |
| 32.831127 | 32.43595 | 21.06069 | 20.913776 |
| 17.83166 | 17.803654 | 17.859222 | 17.84371 |
| 30.709713 | 30.74041 | 18.350698 | 18.481495 |

Plate 2 (Outlier/Flagged Values)

| | | | |
|---|---|---|---|
| 40 | 40 | 40 | 40 |
| 40 | 40 | 40 | 40 |
| 17.807295 | 17.802525 | 22.195833 | 22.12981 |
| 32.831127 | 32.43595 | 21.06069 | 20.913776 |
| 17.83166 | 17.803654 | 17.859222 | 17.84371 |
| 30.709713 | 30.74041 | 18.350698 | 18.481495 |

Plate 3

| | | | |
|---|---|---|---|
| 40 | 40 | 40 | 40 |
| 40 | 40 | 40 | 40 |
| 17.718258 | 17.601076 | 22.291353 | 22.168016 |
| 32.81726 | 32.567535 | 21.084427 | 20.917088 |
| 17.623665 | 17.651794 | 17.852306 | 17.791344 |
| 30.92618 | 30.669481 | 18.361881 | 18.328074 |

Plate 3 (Outlier/Flagged Values)

| | | | |
|---|---|---|---|
| 40 | 40 | 40 | 40 |
| 40 | 40 | 40 | 40 |
| 17.718258 | 17.601076 | 22.291353 | 22.168016 |
| 32.81726 | 32.567535 | 21.084427 | 20.917088 |
| 17.623665 | 17.651794 | 17.852306 | 17.791344 |
| 30.92618 | 30.669481 | 18.361881 | 18.328074 |

Plate 4

| | | | |
|---|---|---|---|
| 40 | 40 | 40 | 40 |
| 40 | 40 | 40 | 40 |
| 17.599699 | 17.502323 | 22.286669 | 22.13237 |
| 32.95936 | 32.51385 | 21.013098 | 20.910128 |
| 17.591288 | 17.577255 | 17.932745 | 17.910442 |
| 30.7185 | 30.56501 | 18.342968 | 18.353554 |

Plate 4 (Outlier/Flagged Values)

| | | | |
|---|---|---|---|
| 40 | 40 | 40 | 40 |
| 40 | 40 | 40 | 40 |
| 17.599699 | 17.502323 | 22.286669 | 22.13237 |
| 32.95936 | 32.51385 | 21.013098 | 20.910128 |
| 17.591288 | 17.577255 | 17.932745 | 17.910442 |
| 30.7185 | 30.56501 | 18.342968 | 18.353554 |

Plate 5

| | | | |
|---|---|---|---|
| 40 | 40 | 40 | 40 |
| 40 | 40 | 40 | 40 |
| 17.6238 | 17.540497 | 22.194326 | 22.169394 |
| 32.441017 | 32.690857 | 21.082005 | 21.014755 |
| 17.461433 | 17.492977 | 17.857912 | 17.853838 |
| 30.795885 | 30.74543 | 18.323055 | 18.409206 |

Plate 5 (Outlier/Flagged Values)

| | | | |
|---|---|---|---|
| 40 | 40 | 40 | 40 |
| 40 | 40 | 40 | 40 |
| 17.6238 | 17.540497 | 22.194326 | 22.169394 |
| 32.441017 | 32.690857 | 21.082005 | 21.014755 |
| 17.461433 | 17.492977 | 17.857912 | 17.853838 |
| 30.795885 | 30.74543 | 18.323055 | 18.409206 |

Plate 6

| | | | |
|---|---|---|---|
| 40 | 37.909855 | 40 | 40 |
| 40 | 40 | 40 | 40 |
| 17.583248 | 17.522343 | 22.233776 | 22.162443 |
| 32.353653 | 32.120243 | 21.099651 | 21.04158 |
| 17.572554 | 17.536425 | 17.900217 | 17.979418 |
| 30.46868 | 30.369146 | 18.377436 | 18.441887 |

Plate 6 (Outlier/Flagged Values)

| | | | |
|---|---|---|---|
| 40 | 37.909855 | 40 | 40 |
| 40 | 40 | 40 | 40 |
| 17.583248 | 17.522343 | 22.233776 | 22.162443 |
| 32.353653 | 32.120243 | 21.099651 | 21.04158 |
| 17.572554 | 17.536425 | 17.900217 | 17.979418 |
| 30.46868 | 30.369146 | 18.377436 | 18.441887 |

Plate 7

| | | | |
|---|---|---|---|
| 40 | 40 | 40 | 40 |
| 40 | 40 | 40 | 40 |
| 17.542034 | 17.644028 | 22.255577 | 22.178228 |
| 32.44262 | 32.117496 | 21.1006 | 21.065472 |
| 17.359932 | 17.583672 | 18.020014 | 17.948322 |
| 30.407469 | 30.394953 | 18.409962 | 18.430319 |

Plate 7 (Outlier/Flagged Values)

| | | | |
|---|---|---|---|
| 40 | 40 | 40 | 40 |
| 40 | 40 | 40 | 40 |
| 17.542034 | 17.644028 | 22.255577 | 22.178228 |
| 32.44262 | 32.117496 | 21.1006 | 21.065472 |
| 17.359932 | 17.583672 | 18.020014 | 17.948322 |
| 30.407469 | 30.394953 | 18.409962 | 18.430319 |

Plate 8

| | | | |
|---|---|---|---|
| 40 | 40 | 40 | 40 |
| 40 | 40 | 40 | 40 |
| 17.552366 | 17.514387 | 22.29376 | 22.219326 |
| 32.712856 | 32.434605 | 21.17902 | 21.133713 |
| 17.521217 | 17.426937 | 18.048174 | 18.03511 |
| 30.526167 | 30.449745 | 18.547752 | 18.469557 |

Plate 8 (Outlier/Flagged Values)

| | | | |
|---|---|---|---|
| 40 | 40 | 40 | 40 |
| 40 | 40 | 40 | 40 |
| 17.552366 | 17.514387 | 22.29376 | 22.219326 |
| 32.712856 | 32.434605 | 21.17902 | 21.133713 |
| 17.521217 | 17.426937 | 18.048174 | 18.03511 |
| 30.526167 | 30.449745 | 18.547752 | 18.469557 |

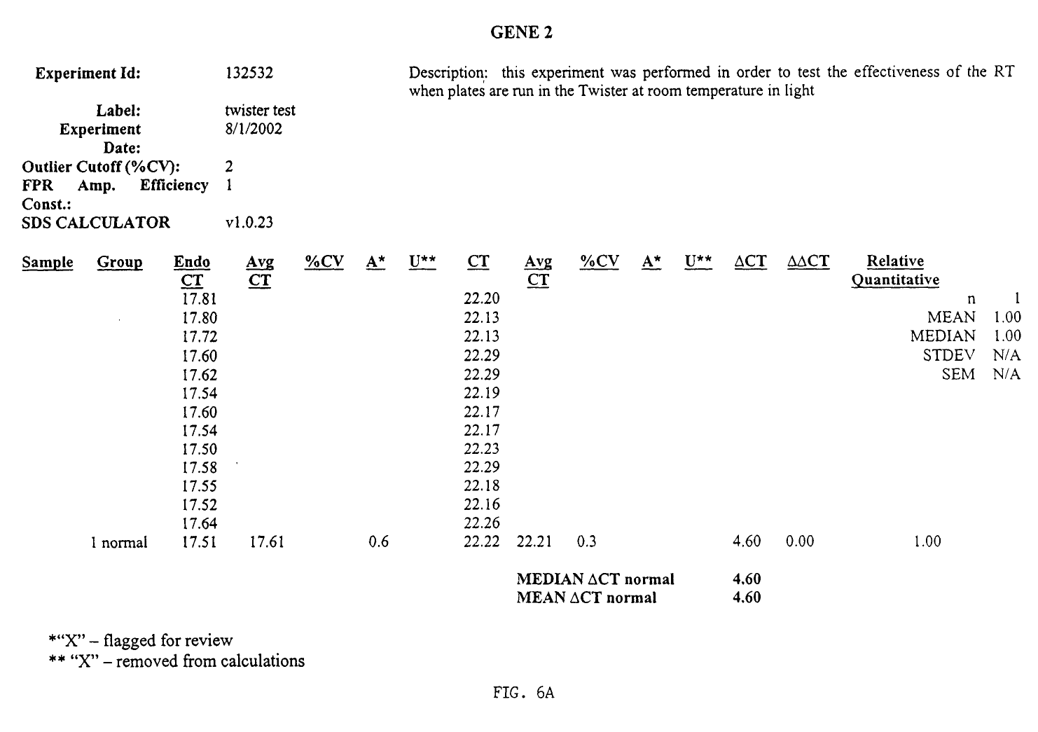

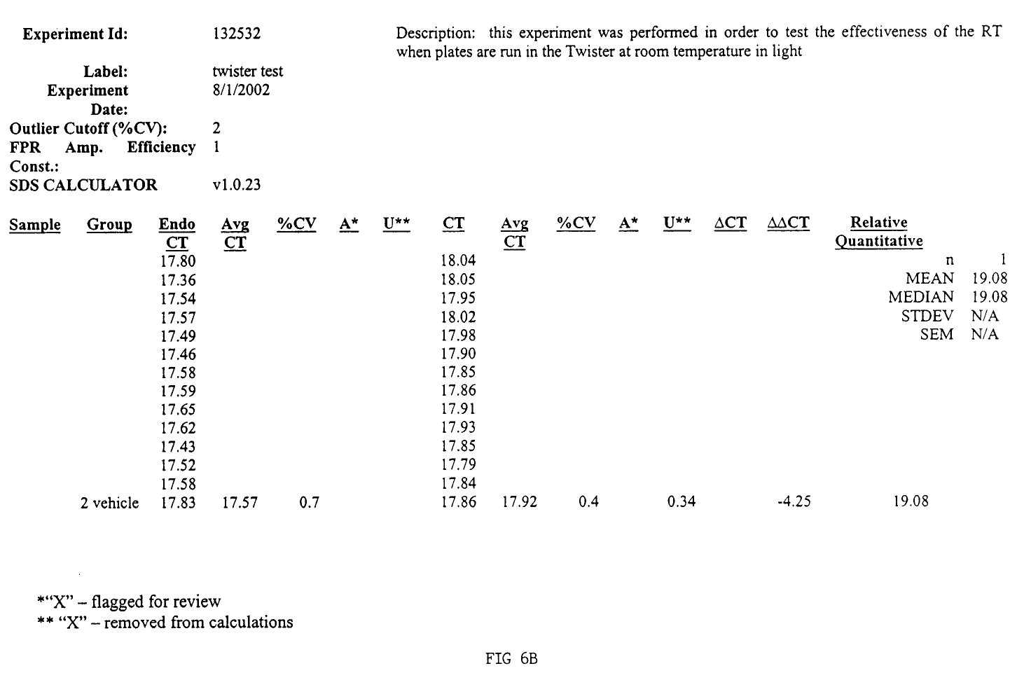

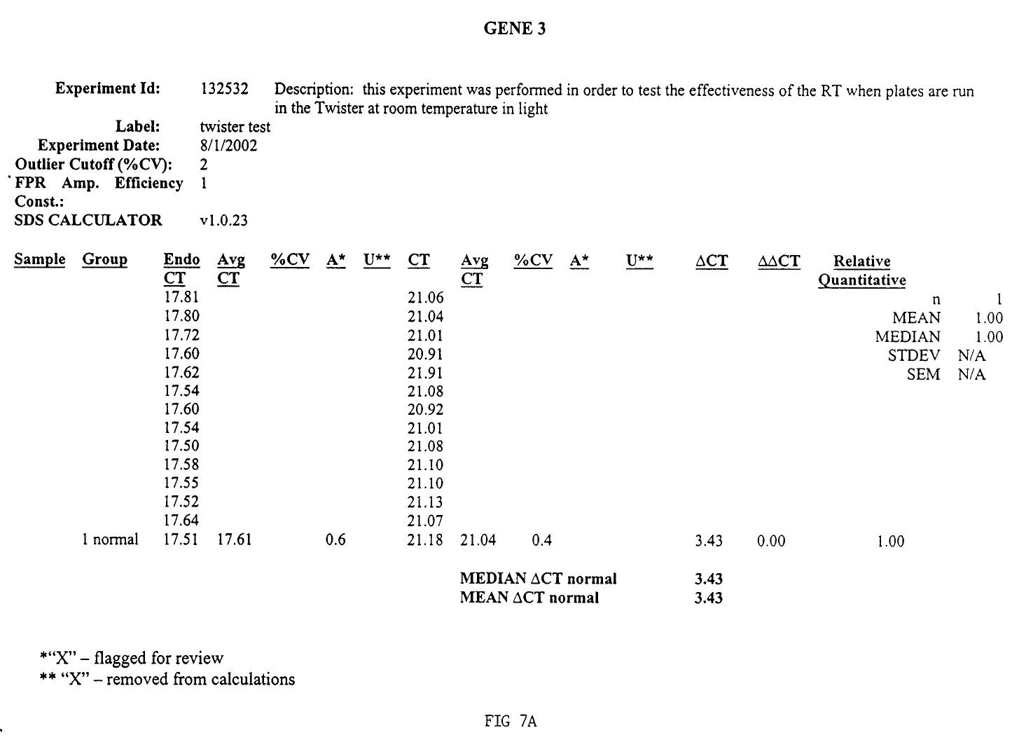

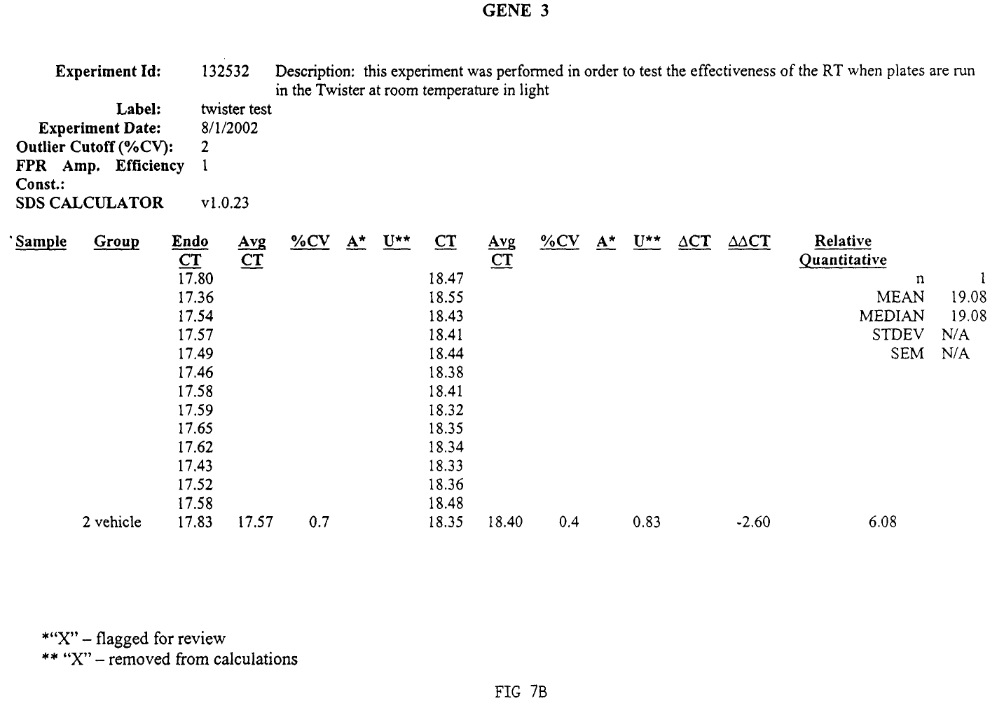

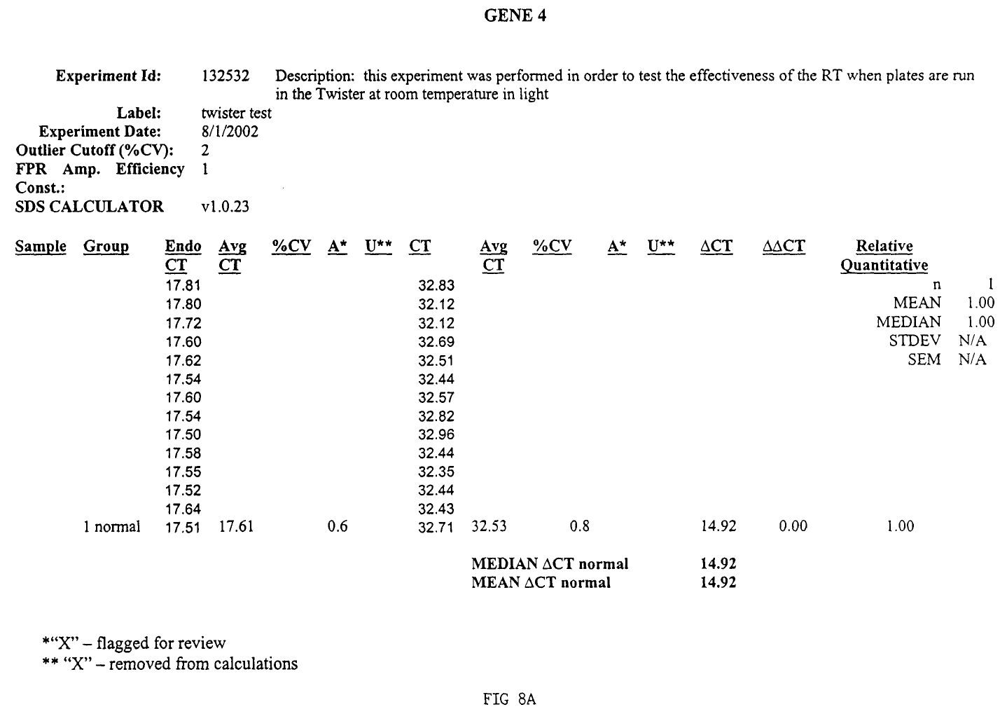

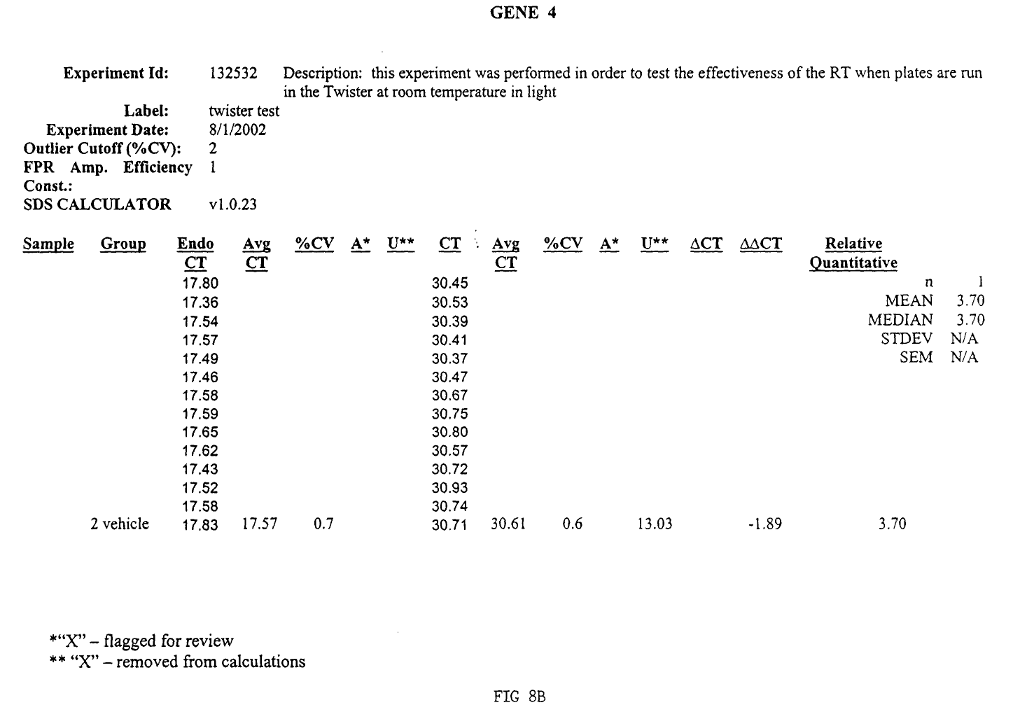

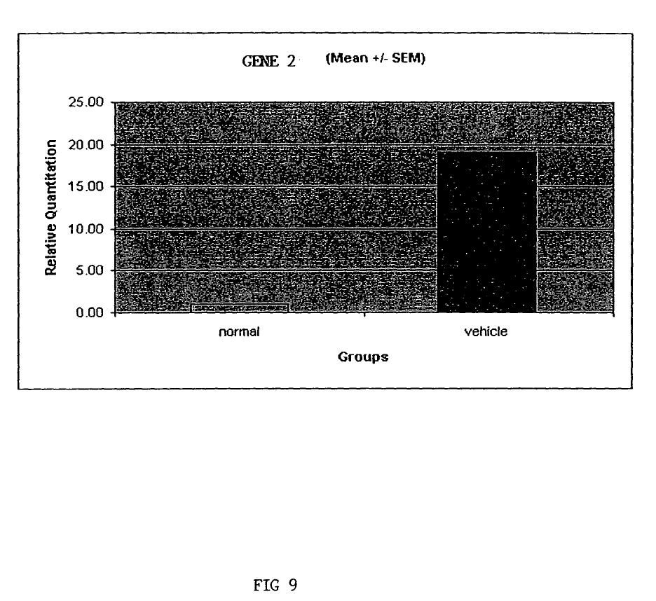

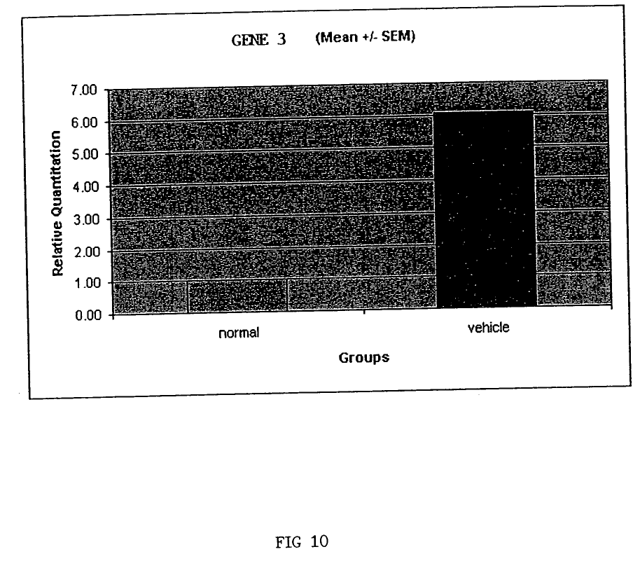

FIGS. 5 through 11 represent data obtained and analyzed in connection with this experiment. While the endogenous control gene (a gene known to have very stable mRNA) maintains the same Ct over a 14 hour room temp window, other genes (Genes 2, 3, and 4) show a mRNA decay that is obvious after 10 hours at room temperature. Initial results indicate that if refrigeration is not available, each gene assayed would have to have room temperature stability determined before TaqMan® experiment was performed. This can be eliminated by refrigeration of the Twister tower containing the TaqMan® plates.

### EXAMPLE 2

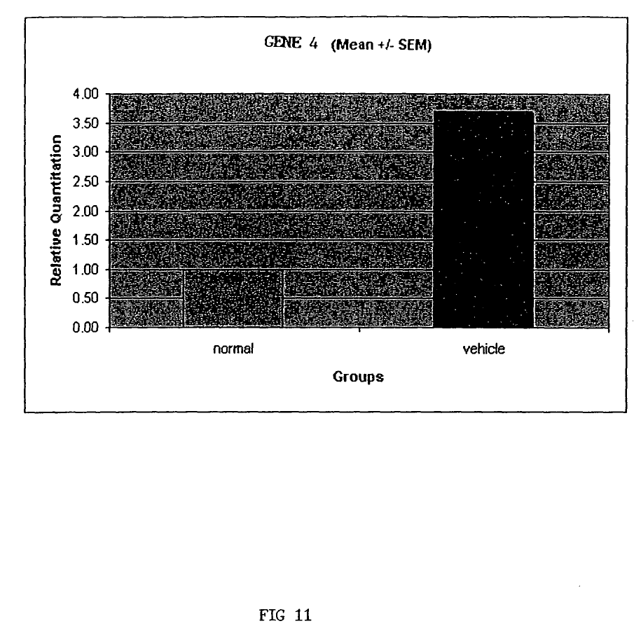

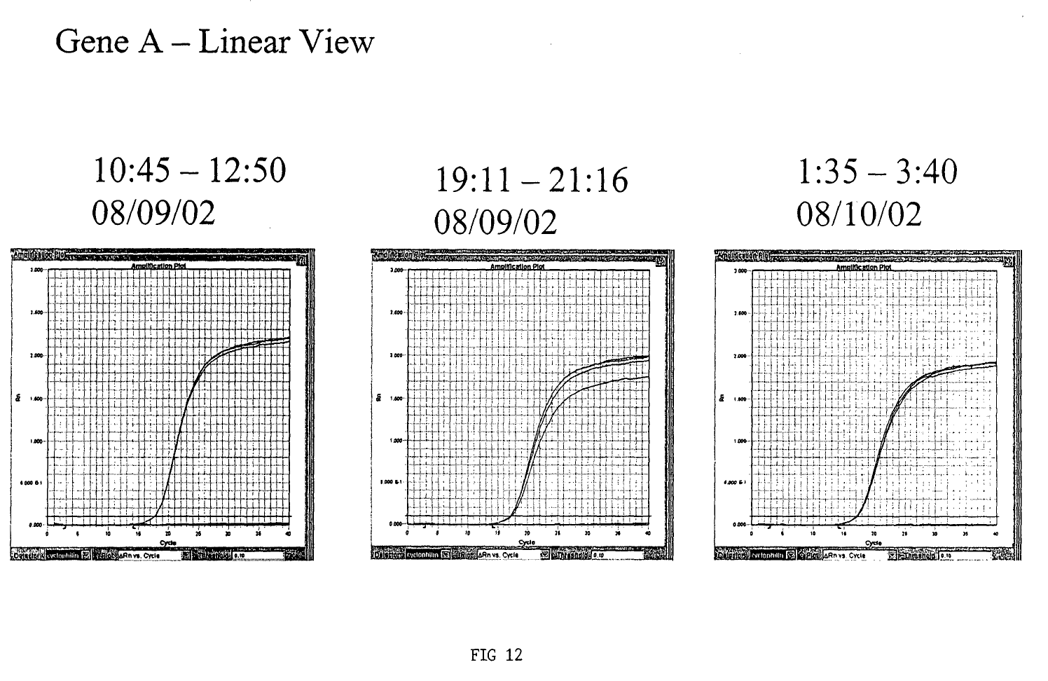

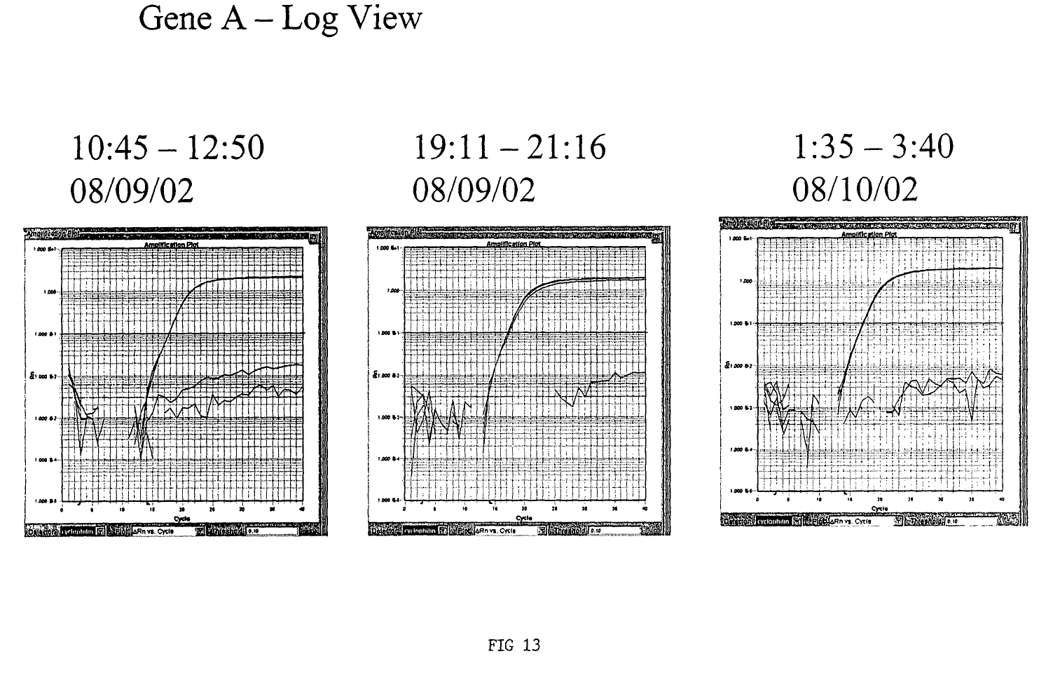

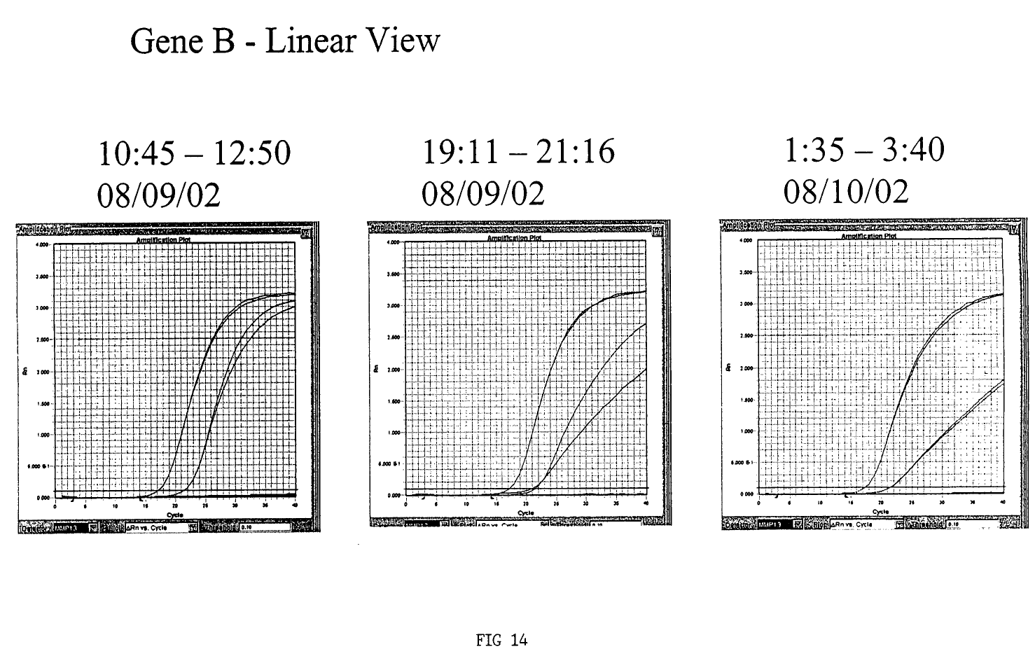

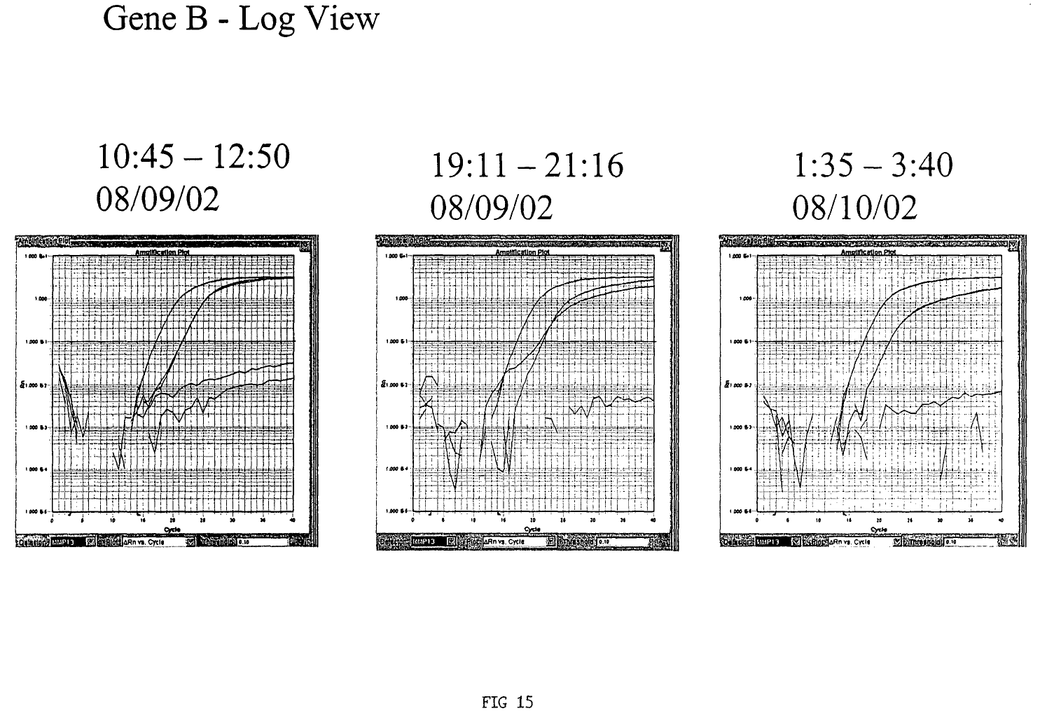

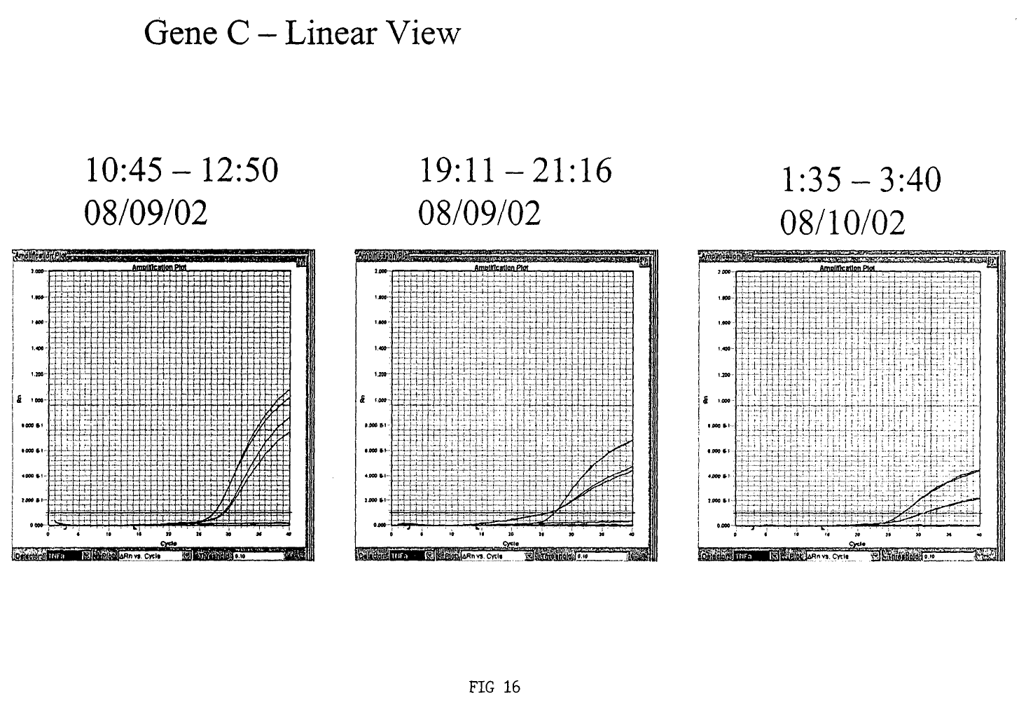

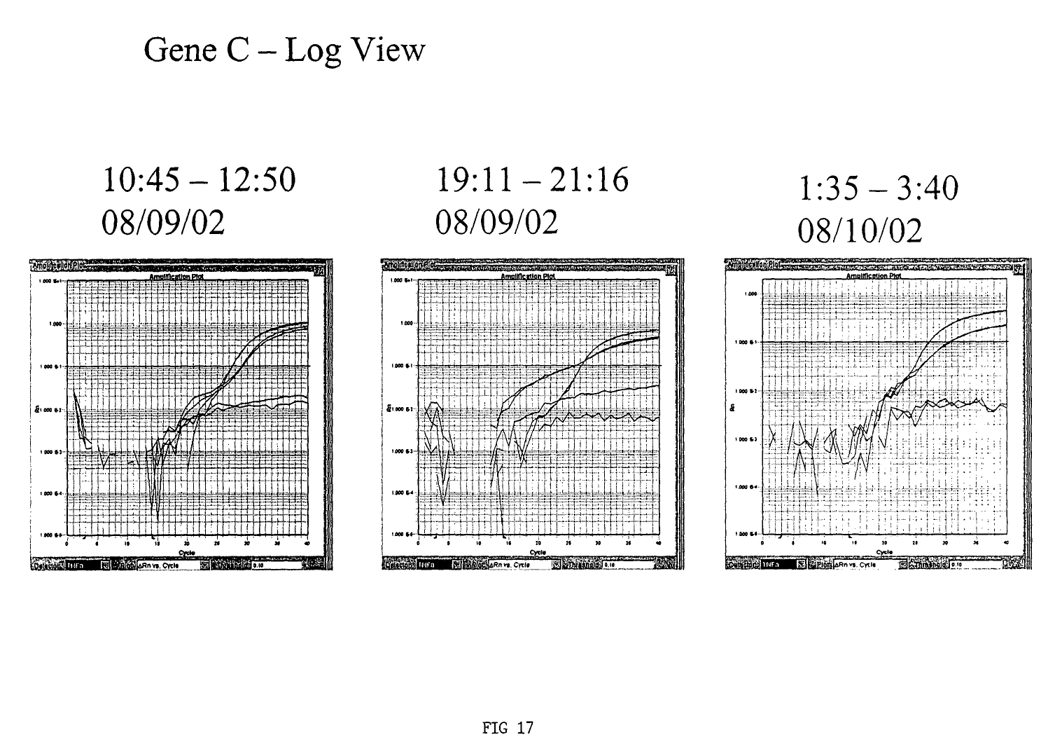

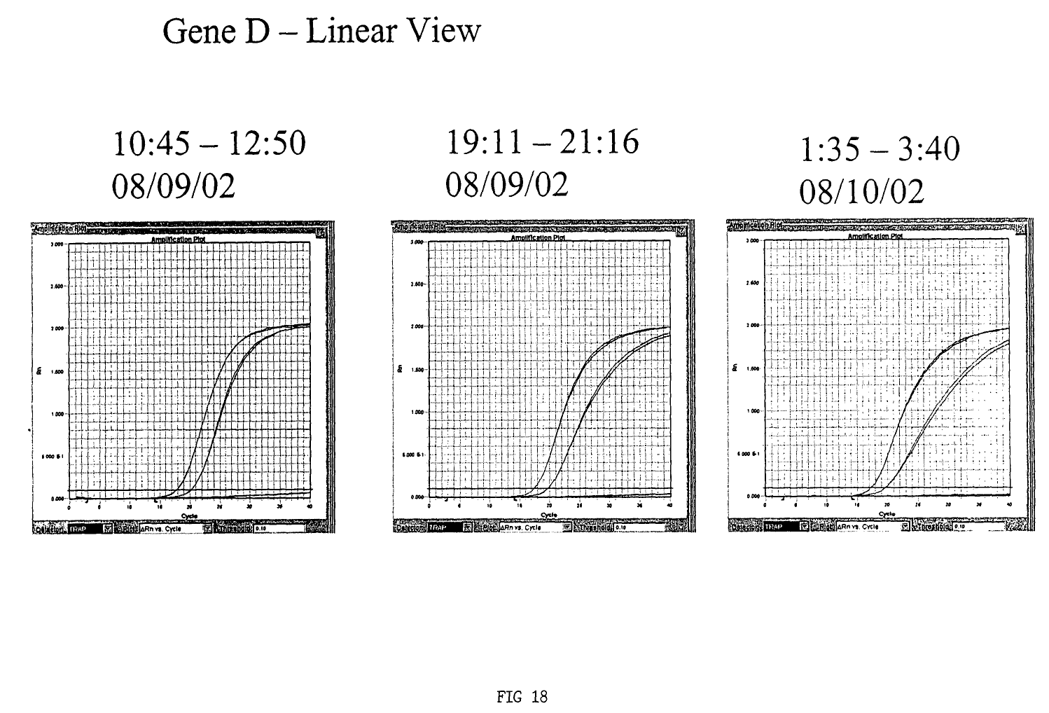

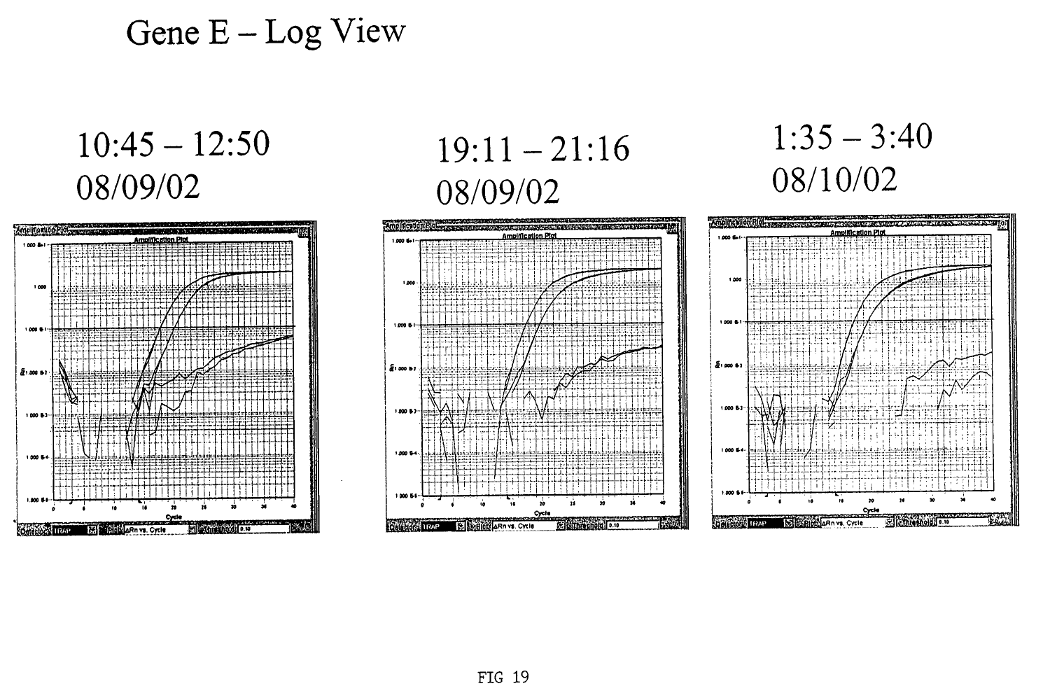

A series of TaqMan® Plates containing were set up to run continuously for several hours and collect data using 4 primer/probe sets and two RNAs, one from a normal rat paw and one from an arthritic rat paw. The last plate was run the next morning approximately 15 hours later. In addition, there was a third run between these two tests. As shown in FIGS. 11 through 19, data from Gene A and Gene D of the four genes (Gene A, Gene B, Gene C, Gene D) is fairly stable. However, as indicated by the height of the curves (the delta Rn), data from the primer/probe set of Gene B begins to show signs of failing and the primer probe set of Gene C deteriorates with time. When the height of the curves decreases with time, a lack of robustness of the assay is indicated.

The deterioration of the data is believed to be a result of the following: (a) the primer dimers are forming in the reaction since room temperature would be appropriate for annealing; (b) exposure to light while plates are sitting in the queue causes degradation of the probe and release of fluorescent dye, increasing background fluorescence and decreasing overall strength of signal (delta Rn); (c) variability of RNA and RNA stability from one preparation of RNA to the next; and (d) overall effectiveness of the primer/probe set since some primer/probe sets are more sensitive and robust than others.

Although making and using various embodiments of the present invention have been described in detail above, it should be appreciated that the present invention provides many applicable inventive concepts that can be embodied in a wide variety of specific contexts. The specific embodiments discussed herein are merely illustrative of specific ways to make and use the invention, and do not delimit the scope of the invention. Those skilled in the art will recognize that changes in the apparatus and process may be made without departing from the spirit of the invention. Such changes are intended to fall within the scope of the following claims.

It is to be understood that the disclosed embodiments are merely exemplary of the invention that may be embodied in various and alternative forms. The figures are not necessarily to scale where some features may be exaggerated or minimized to show details of particular components. Therefore, specific structural and functional details disclosed herein are not to be interpreted as limiting, but merely as a basis for the claims and as a representative basis for teaching one skilled in the art to variously employ the present invention.

We claim:

1. A metal block for use in a high throughput RNA laboratory comprising:
a plurality of wells, each said well having an open cylindrical upper end and a closed conical lower end, each said well accommodating a biological sample receptacle having substantially the same shape as said well, wherein each said well maintains the temperature of a biological sample in the receptacle during sample set-up and prior to reverse transcriptase and polymerase chain reaction analysis.

2. The metal block of claim 1 wherein said metal is aluminum.

3. The metal block of claim 1 wherein the biological sample receptacles are microtubes.

4. The metal block of claim 1 wherein the biological sample receptacles are comprised within a 96-well plate.

5. The metal block of claim 1 wherein the biological sample receptacles are comprised within a 384-well plate.

6. The metal block of claim 1 wherein the temperature maintained is below room temperature.

7. A metal block for an automated liquid handling device used in genetic analysis comprising:
a plurality of wells, each said well having an open cylindrical upper end and a closed conical lower end, each said well accommodating a biological sample receptacle having substantially the same shape as said well wherein the temperature of a biological sample in the receptacle is maintained.

8. The metal block of claim 7 wherein said metal is aluminum.

9. The metal block of claim 7 wherein the biological sample receptacles are microtubes.

10. The metal block of claim 7 wherein the biological sample receptacles are comprised within a 96-well plate.

11. The metal block of claim 7 wherein the biological sample receptacles are comprised within a 384-well plate.

12. The metal block of claim 7 wherein the automated liquid handling device comprises a surface being maintained below room temperature.

13. The metal block of claim 7 wherein the temperature maintained is below room temperature.

14. An apparatus for an automated liquid handling device used in genetic analysis comprising:
a plurality of biological sample receptacles; and
a metal block comprising a plurality of wells, each said well having an open cylindrical upper end and a closed conical lower end, each said well accommodating said biological sample receptacle having substantially the same shape as said well, wherein each said well maintains the temperature of a biological sample in the receptacle.

15. An improved automated liquid handling device for genetic analysis comprising:
a metal block comprising a plurality of wells, each said well having an open cylindrical upper end and a closed conical lower end, each said well accommodating a biological sample receptacle having substantially the same shape as said well, wherein each said well maintains the temperature of a biological sample in the receptacle during sample set-up and prior to polymerase chain reaction analysis.

16. An improved automated liquid handling device for genetic analysis of biological samples, the handling device having a table, a pod for transferring fluid to a well located on the table and a means for moving the pod relative to the table between selected locations on said table, wherein the improvement comprises:
a metal block comprising a plurality of wells, each said well having an open cylindrical upper end and a closed conical lower end, each said well accommodating a biological sample receptacle having substantially the same shape as said well, wherein the temperature of a biological sample in the receptacle during sample set-up and prior to polymerase chain reaction analysis is maintained.

17. An apparatus for high throughput RNA analysis of a biological sample comprising:
a metal block comprising a plurality of wells, each said well having an open cylindrical upper end and a closed conical lower end, each said well accommodating a biological sample receptacle having substantially the same shape as said well, wherein each said well maintains the temperature of the biological sample in the receptacle;
an automated liquid handling device; and
a PCR amplification device wherein the biological sample is inserted into the receptacles of said wells of said metal block by said automated liquid handling device and said PCR amplification device causes reverse transcriptase polymerase chain reaction to determine the presence of RNA or DNA.

18. The apparatus of claim 17 wherein said reverse transcriptase polymerase chain reaction is one step.

19. The apparatus of claim 17 wherein said reverse transcriptase polymerase chain reaction is two steps.

20. A method of handling a liquid biological sample in a high throughput RNA laboratory, comprising the steps of:
chilling a metal block, the metal block having a plurality of wells, each well having an open cylindrical upper end and a closed conical lower end, each well accommodating a biological sample receptacle having substantially the same shape as the well, wherein the temperature of the biological sample is maintained;
inserting the biological sample receptacle into the metal block;
positioning the metal block onto an automated liquid handling device; and
transferring the biological sample into biological sample receptacle in the metal block for reverse transcriptase and polymerase chain reaction analysis.

21. A method of handling a liquid biological sample in a high throughput RNA laboratory, comprising the steps of:
cooling a metal block, the metal block having a plurality of wells, each well having an open cylindrical upper end and a closed conical lower end, each well accommodating a biological sample receptacle having substantially the same shape as the well, wherein the temperature of the biological sample is maintained;
inserting the biological sample receptacle into the metal block;
positioning the metal block onto an automated liquid handling device; and
transferring the biological sample into biological sample receptacle in the metal block for reverse transcriptase and polymerase chain reaction analysis.
